(19) United States
(12) Patent Application Publication
(10) Pub. No.: US 2014/0012220 A1
(43) Pub. Date: Jan. 9, 2014

(54) Protective Underwear With Absorbent Insert

(71) Applicant: Chana Flieg, Ashburn, VA (US)

(72) Inventor: Chana Flieg, Ashburn, VA (US)

(21) Appl. No.: 13/927,234

(22) Filed: Jun. 26, 2013

Related U.S. Application Data
(60) Provisional application No. 61/669,078, filed on Jul. 8, 2012.

(51) Int. Cl. A61F 13/49

(52) U.S. Cl. CPC A61F 13/49006
USPC 604/385.14

(57) ABSTRACT

The present invention provides protective underwear including shorts and removable absorbent inserts that can be pulled away from and attached to the shorts while the shorts are pulled down but not completely removed. This allows a soiled insert to be removed, and a clean replacement insert to be installed, without having to remove the person's pants and shoes. The assembled protective underwear also looks like normal underwear from the outside and is sufficiently less bulky than a traditional adult diaper allowing it to be effectively concealed when worn under most clothing. This improvement in discreetness and convenience can make a major difference to the user, such as child or adult hoping to reduce potential embarrassment while maintain maximum levels of activity.

### REFERENCE TO RELATED APPLICATION

This application claims priority to U.S. Provisional Patent Application Ser. No. 61/669,078 entitled “Protective Underwear Device” filed Jul. 8, 2012, which is incorporated by reference.

### TECHNICAL FIELD

The present invention relates to incontinent devices and, more particularly, to protective underwear with removable absorbent inserts providing more discreet, convenient and cost effective protective undergarments.

### BACKGROUND

Varying degrees of incontinence can have a variety of causes. While diapers are suitable for infants and adult diapers are suitable for older persons in some situations, these products have significant drawbacks for certain individuals. In particular, there is no good solution presently available for children beyond potty training age who still require some degree of incontinence protection. While infant diapers are generally too small and adult diapers are generally too large, typical pad inserts have an insufficient capacity and are prone to coming dislodged too easily to provide a sufficiently secure solution. Importantly for these individuals, even a single event of public loss of protection can be emotionally traumatic.

While diapers are effective at preventing leakage, they are sufficiently bulky to be readily recognized when worn under typical clothing. Wearing a diaper that is readily recognized by casual observers can be embarrassing by itself and the problem can be seriously exacerbated whenever changing clothes is required in a group environment, such as in a locker room for gym class or a public pool. Teasing can be relentless among adolescents, which can increase social adjustments and developmental challenges for those children who are already dealing with extraordinary challenges. These difficulties can follow the affected children into adulthood, where similar problems can be experienced in the workplace and health club. And, of course, anyone may develop issues with incontinence at any stage of life, whether arising from accident, injury, disease or another cause.

Another significant drawback with traditional adult diapers, adult disposable pants, or children's “pull-ups” is the need for the user to completely remove their pants and shoes in the ordinary course of use in order to remove and replace the diaper. This can make for an embarrassing event whenever the diaper needs to be changed, particularly for those individuals who can use the commode independently but require assistance to remove and replaced their shoes and pants. This may the case, for example, with for many individuals in wheelchairs. A significant loss of independence and dignity can therefore be felt by those who require such assistance, sometimes to the point where they may choose not to participate in potentially constructive activities in which they would otherwise participate.

As a result, there is a persistent need for a more discreet and convenient protective undergarment solution and, more particularly, for a protective underwear solution for active persons that can be used without requiring complete removal of the person's pants and shoes in the ordinary course of use.

### SUMMARY OF THE INVENTION

The present invention meets the needs described above in protective underwear including shorts and removable absorbent inserts that can be pulled away from and attached to the shorts while the shorts are pulled down but not completely removed. This allows a soiled insert to be removed, and a clean replacement insert to be installed, without having to remove the person's pants and shoes. The assembled protective underwear also looks like normal underwear from the outside and is sufficiently less bulky than a traditional adult diaper to be effectively concealed when worn under most clothing. This improvement in discreetness and convenience can make a major difference to the user, such as child or adult hoping to reduce potential embarrassment while maintain maximum levels of activity.

The protective underwear typically included a conventional pair of shorts (e.g., boy's briefs or girl's panties) with an optional waterproof coating applied to the inside surface. Fasteners, such as snaps or hook and loop (Velcro), are also affixed to the inside surface of the shorts for removable attachment to an absorbent liner. The fasteners, such as snaps, may be sewn directly to a backing strip which is sewn to the shorts with appropriate colored thread to help conceal the fasteners as seen from outside the shorts. The removable absorbent liner includes corresponding fasteners on the outside surface allowing the liner to be selectively attached to, and removed from, the shorts while the shorts are pulled down but not completely removed from the wearer. The liner may also include elastic on each side of the crotch area to minimize the chance of leakage. The liner is much more substantial and secure than a traditional undergarment pad insert, while being much smaller and more discreet than a traditional diaper. The protective underwear is also more cost effective than current product as the liner requires significantly less material than a traditional diaper, while it provides far greater security than a conventional pad insert and results in fewer destroyed pairs of shorts.

In view of the foregoing, it will be appreciated that the present invention provides improved protective underwear with a secure yet removable absorbent liner that can be removed and replaced while the shorts are pulled down but not completely removed from the wearer. The specific structures and techniques for accomplishing the advantages described above will become apparent from the following detailed description of the embodiments and the appended drawings and claims.

### BRIEF DESCRIPTION OF THE DRAWINGS

FIG. 1 is a front perspective view of the protective underwear.

FIG. 2 is a front perspective view of the protective underwear opened to reveal the absorbent insert on the inside of the shorts.

FIG. 3 is a top perspective view of the protective underwear opened to reveal the absorbent insert on the inside of the shorts.

FIG. 4 is a top perspective view of the protective underwear with the absorbent insert removed to reveal the fasteners on the inside of the shorts.

FIG. 5 is a top perspective view of the inside of the removable liner.

FIG. 6 is a top perspective view of the outside of the removable liner.

FIG. 7 is an exploded view of the protective underwear.

FIG. 8 is a front perspective view of the protective with an alternative type of shorts and an alternative type of fastener.

FIG. 9 is a front perspective view of the removable liner with multiple fasteners for use with different sizes of shorts.

FIG. 10 is a front perspective view of the shorts with multiple fasteners for use with different sizes of inserts.

### DETAILED DESCRIPTION OF THE EMBODIMENTS

The present invention may be embodied in protective underwear including shorts and removable absorbent inserts that can be pulled away from and attached to the shorts while the shorts are pulled down but not completely removed. This allows a soiled insert to be removed, and a clean replacement insert to be installed, without having to remove the person's pants and shoes. The shorts may be any variety of fitted underwear, such as boy's briefs or girl panties. Simple fasteners, such as snaps or hook and loop (Velcro) strips are included to the inside of the shorts and the outside of the liner to enable the liner to the attached and removed as desired. Multiple rows or strips of fasteners may be provided on the shorts to accommodate liners of different sizes. A flexible waterproof coating may be applied to the inner side of the shorts, such as spray-on latex, urethane or other suitable polymer. A latex-free coating may be preferred due to the prevalence of latex allergies. The coating may be omitted, if desired, and different models of shorts may be provided with different types of coatings to accommodate persons with different allergy needs.

The removable absorbent pad or liner includes corresponding fasteners on the outside surface allowing the liner to be selectively attached to, and removed from, the shorts while the shorts are pulled down but not completely removed from the wearer. The liners are preferably manufactured specifically for the protective underwear device, but may be adapted (cut and sewn) from a conventional diaper if a properly fashioned liner is not available in the market. The material and layer technology used in diapers is directly applicable to the present liners, which may be thought of essentially as the center portion of the diaper, trimmed to size for this application, and augmented with fasteners, to produce a selectively removable and properly sized insert for the shorts. The fasteners, such as snaps or hook and loop (Velcro) strips, are preferably sewn directly to the liner to provide a secure attachment. The liner may also include elastic on each side of the crotch area to minimize the chance of leakage.

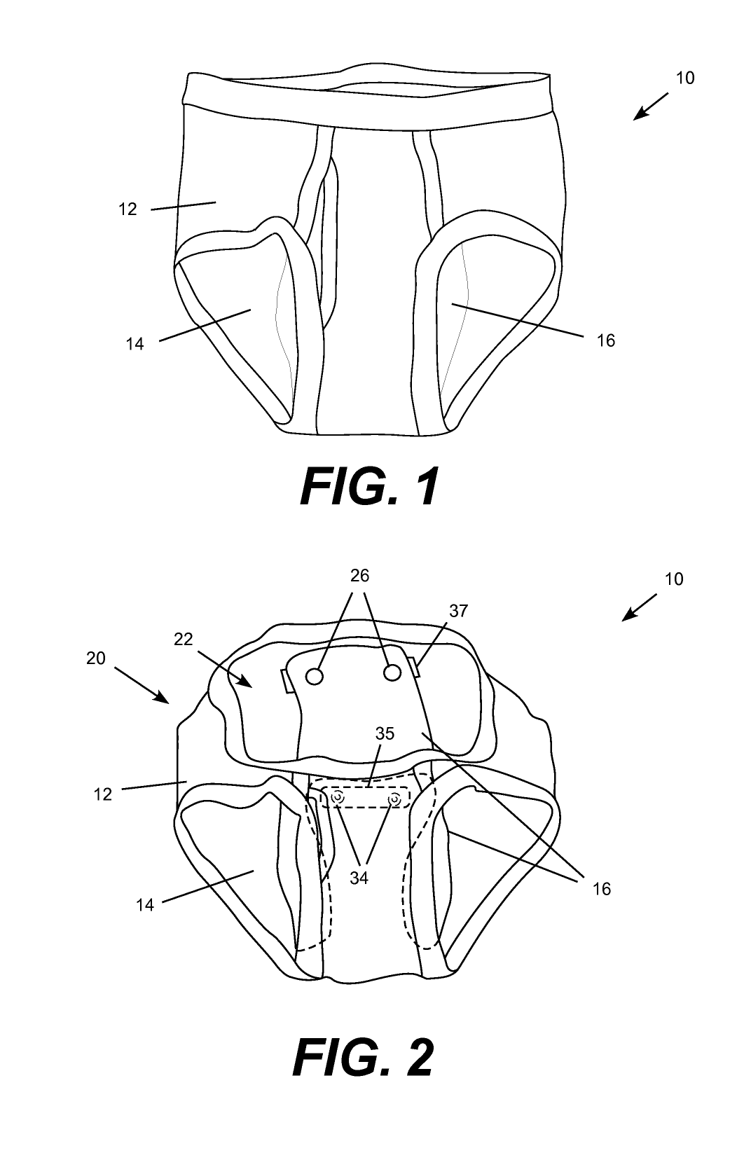

Turning now to the figures, FIG. 1 is a front perspective view of the protective underwear 10 including a pair of shorts 12. In this embodiment the shorts are a pair of boy's briefs, but the invention may utilize any type of fitted garment, such as a bodysuit, bathing suit, pantyhose, and so forth. The shorts are optionally coated with a waterproof layer 14, such as spray-on urethane, latex or other suitable material. The absorbent liner 16 is sized to fit the shorts, making the liner barely visible in this view and when worn in the usual manner.

FIG. 2 is a front perspective view of the protective underwear 10 opened to reveal the absorbent insert 16 on the inside of the shorts 12. The protective underwear includes a shorts assembly 20 and an insert assembly 22, where the insert assembly is selectively attachable to the shorts assembly. The shorts assembly 20 includes the standard shorts 12 or other base garment with the optional waterproof coating 14 and fasteners attached on the front and rear inner surfaces. In this embodiment, the front fasteners 34 are sewn to a backing strip 35, which is sewn to the shorts with an appropriate colored thread to help conceal the fasteners as viewed from outside the shorts. Similarly, the rear fasteners are sewn to a backing strip 37, which is sewn to the shorts. The insert assembly 22 includes the basic liner 16 with fasteners attached on the front and rear outer surfaces (the fasteners 26 are shown in FIG. 2) and may also include elastic bands on the edges in the crotch area to improve form fitting of the liner and minimize the chance for leakage. The liner 16 extends from just under the waistband at the front of the shorts, down and across the crotch area, and up on the rear of the shorts to just under the waistband at the rear of the shorts.

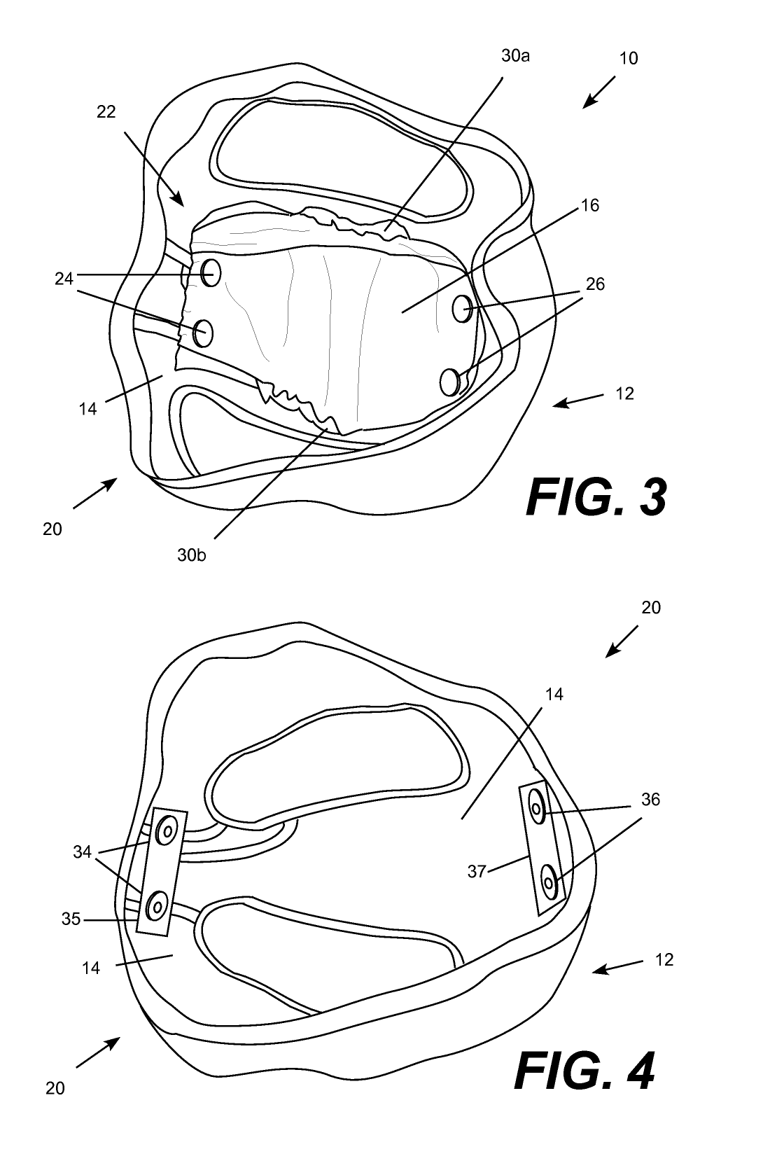

FIG. 3 is a top perspective view of the protective underwear 10 opened to reveal the absorbent insert 12 on the inside of the shorts. In this view, the backing of the snaps 24, 26 on the liner are shown along with the elastic bands 30a-b on the edges of the liner in the crotch area to improve form fitting of the liner. FIG. 4 shows the corresponding view with the liner assembly removed from the shorts assembly revealing the front snaps 34 attached to the backing strip 35 the rear snaps 36 attached to the backing strip 37 on the inside of the shorts 12. The shorts assembly 20 holds together as a first unit, while the liner assembly 22 holds together as a second unit, providing a convenient two-part underwear product in which the liner assembly can be easily attached to and removed from the shorts assembly while the user's pants are pulled down but not fully removed.

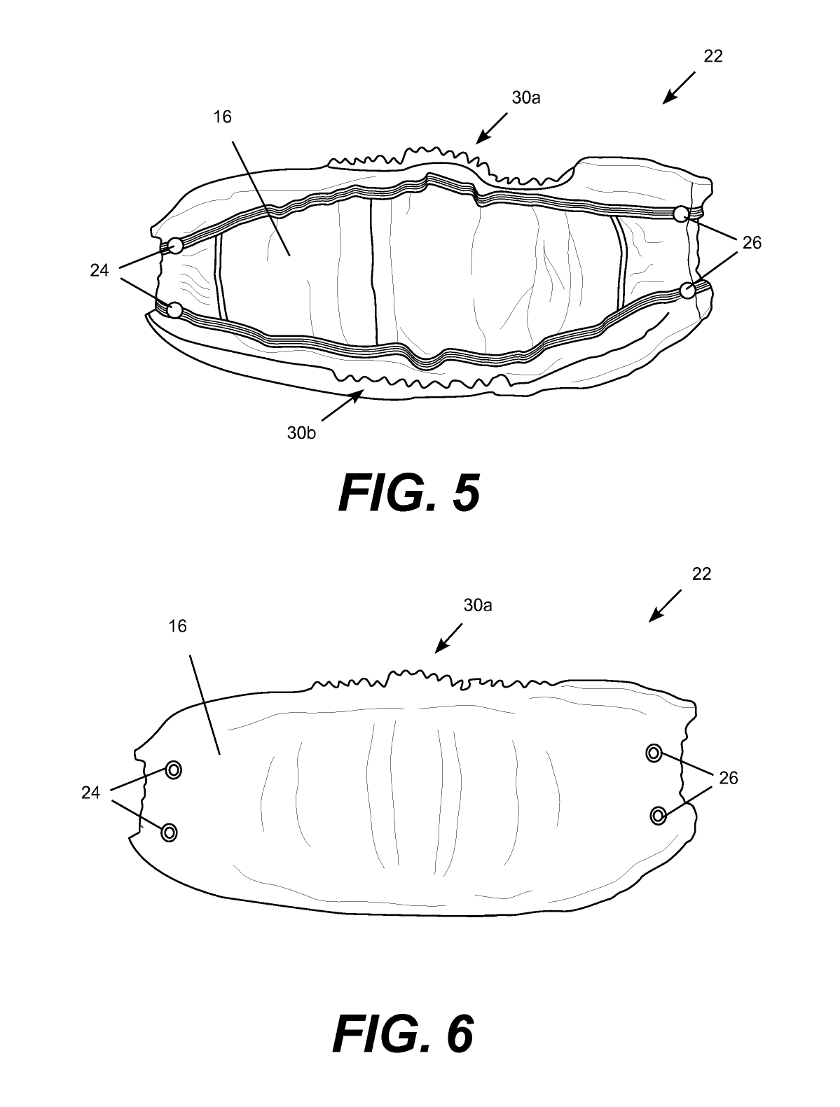

FIG. 5 is a top perspective view of the inside of the removable liner assembly 22, while FIG. 6 is a corresponding view of the outside of the liner assembly. The liner snaps 24, 26 may extend through the liner with the backings visible on the inside view of FIG. 5. The operative sides (stud or socket) of the snaps are visible on the outside view of FIG. 6. Backings for the snaps improve the security of the attachment of the snaps to the liner but may be omitted if desired. The elastic bands 30a-b on the edges are also shown in these views.

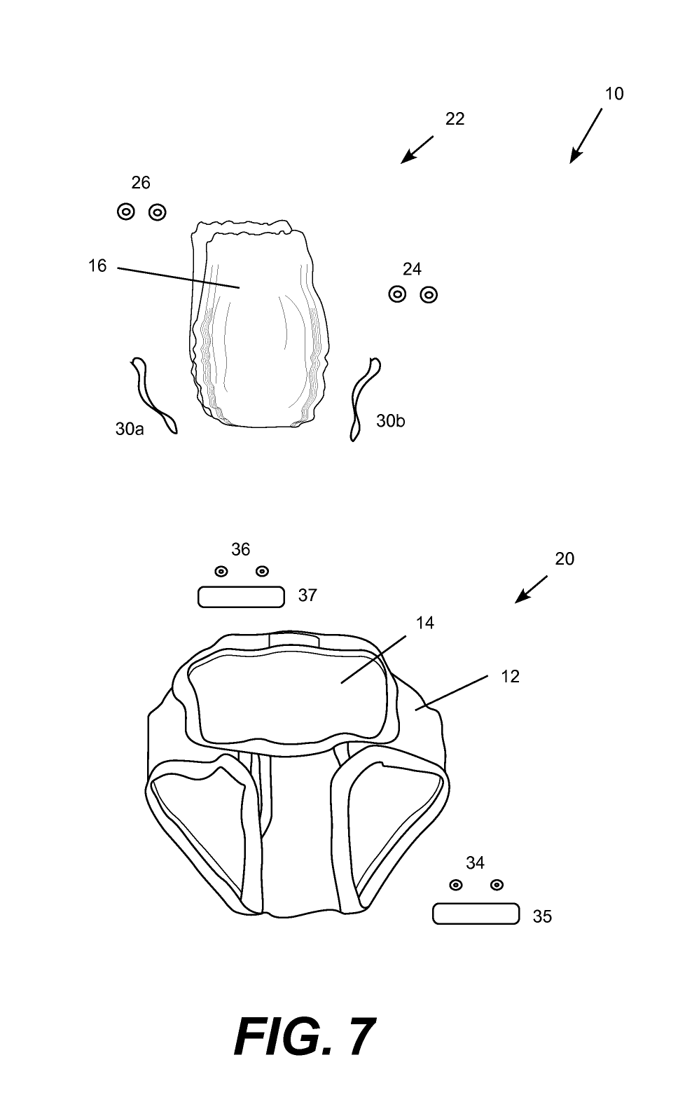

FIG. 7 is an exploded view of the protective underwear 10. The shorts assembly 20 consists essentially of the standard shorts 12 or other base garment, the optional waterproof coating 14, the front snaps 34 attached to the backing strip 35 the rear snaps 36 attached to the backing strip 37 on the inside of the shorts 12. The snaps on the shorts are preferably attached to the sturdy fabric backing strip to withstand daily wear and tear from detaching the insert. This fabric backing strip also makes the snaps nearly undetectable from the outer view of shorts. The operative side of the snap on the shorts may be the stud or socket side, as a matter of design choice. The liner assembly includes the basic liner 16, the snaps 24, 26 and the elastic bans 30a-b. Again, the operative side of the snap on the liner may be the stud or socket side, and a backing may be included, as matters of design choice.

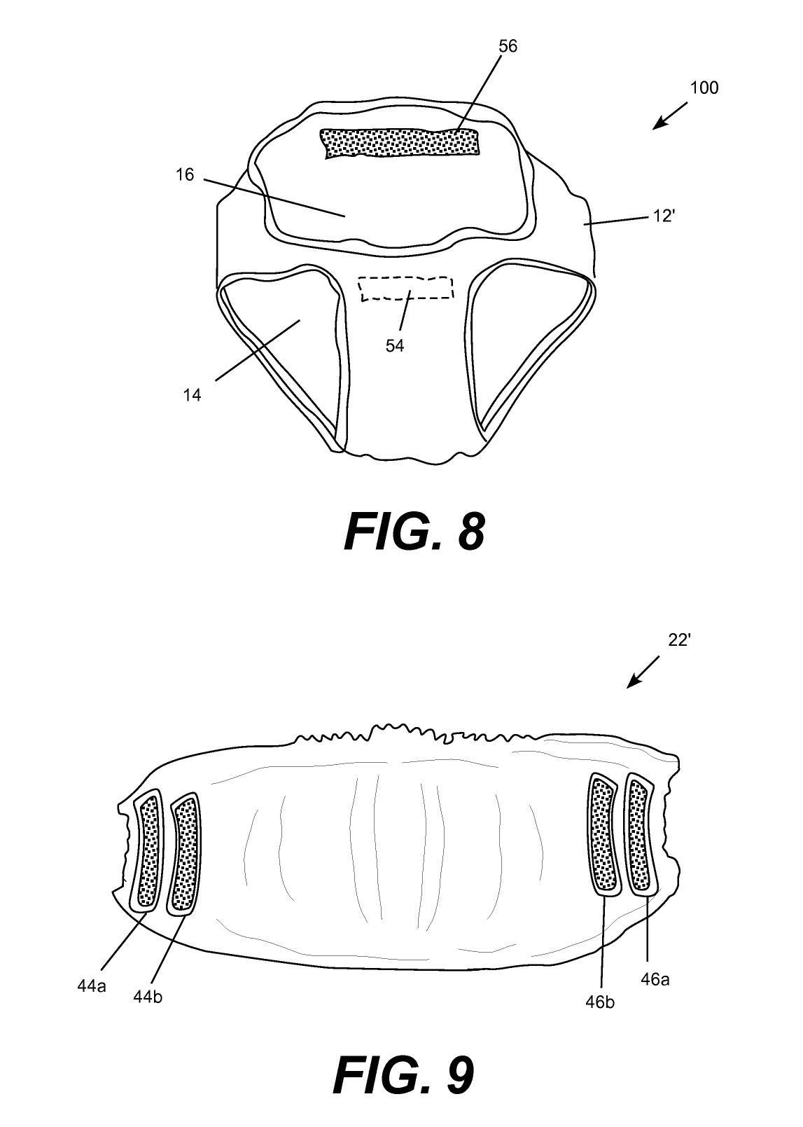

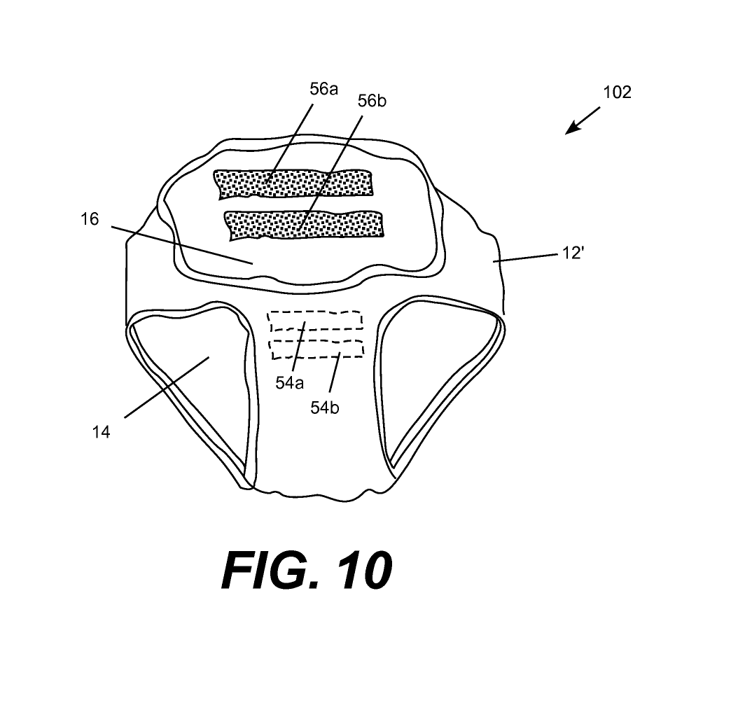

FIG. 8 is shows an alternative embodiment of the protective underwear 100, in this case using a girl's panties as the shorts 12′ as the example base garment. This alternative utilizes hook and loop (Velcro) strips 54, 66 rather than snaps as the fasteners. FIG. 9 shows the corresponding liner 22′, which in this alternative includes multiple rows of the hook and loop fasteners 44a-b and 46a-b. Having multiple rows of fasteners on the liner allows the same liner to be used with different sizes of shorts. Unused fasteners may be removed before use, if desired. FIG. 10 shows another alternative 102, in which the shorts include multiple rows of fasteners 54a-b and 56a-b for use with different sizes of inserts. Again, unused fasteners may be removed before use, if desired.

Those skilled in the art will appreciate that the foregoing describes preferred embodiments of the invention and that many adjustments and alterations will be apparent to those skilled in the art within the spirit and scope of the invention as defined by the appended claims.

The invention claimed is:

1. A protective underwear, comprising:
a shorts assembly comprising a pair of shorts having an inside surface and fasteners affixed to the inside surface; and
a liner assembly comprising an absorbent liner having an outside surface, edges in a crotch area, fasteners affixed to the outside surface, and elastic bands along the edges in the crotch area;
wherein the fasteners on the absorbent liner are positioned for selective attachment to the fasteners on the shorts for selective attachment of the liner to the inside surface of the shorts; and
wherein the liner assembly is sized to be substantially located within the shorts when attached to the inside surface of the shorts.

2. The protective underwear of claim 1, further comprising a waterproof coating on the inside surface of the shorts.

3. The protective underwear of claim 1, further comprising a backing strip wherein one or more of the fasteners are sewn to the backing strip and the backing strip is sewn to the shorts.

4. The protective underwear of claim 3, wherein the fasteners comprise snaps.

5. The protective underwear of claim 1, wherein the fasteners comprise hook and loop fasteners.

6. The protective underwear of claim 1, wherein the liner is configured for removal from the shorts while the shorts are worn by the user wearing while the pants are pulled down but not removed from the user.

7. A method for providing incontinence protection, comprising the steps of:
obtaining a protective underwear including a shorts assembly comprising a pair of shorts having an inside surface and fasteners affixed to the inside surface, and a liner assembly comprising an absorbent liner having an outside surface and edges in a crotch area with fasteners affixed to the outside surface and elastic bands along the edges in the crotch area;
attaching the liner assembly to the shorts assembly with the fasteners on the absorbent liner attached the fasteners on the shorts;
wearing the protective underwear under pants; and
in response to detecting a soiled condition of the liner, pulling down but not removing the pants, pulling down but not removing the protective underwear, removing the liner assembly from the shorts assembly, attaching a replacement liner assembly to the shorts, pulling up the protective underwear, and pulling up the pants.

8. The method of claim 7, further comprising the step of obtaining the protective underwear with a waterproof coating on the inside surface of the shorts.

9. The method of claim 8, further comprising the step of obtaining the protective underwear with a backing strip wherein one or more of the fasteners are sewn to the backing strip and the backing strip is sewn to the shorts.

10. A protective underwear, consisting essentially of:
a shorts assembly comprising a pair of shorts having an inside surface and fasteners affixed to the inside surface;
a liner assembly comprising an absorbent liner having an outside surface, edges in a crotch area, and fasteners affixed to the outside surface;
wherein the fasteners on the absorbent liner are positioned for selective attachment to the fasteners on the shorts for selective attachment of the liner to the inside surface of the shorts; and
wherein the liner assembly is sized to be substantially located within the shorts when attached to the inside surface of the shorts.

11. The protective underwear of claim 10, further comprising a backing strip wherein one or more of the fasteners are sewn to the backing strip and the backing strip is sewn to the shorts.

12. The protective underwear of claim 11, wherein the fasteners comprise snaps.

13. The protective underwear of claim 10, further comprising elastic bands along the edges in the crotch area.

14. The protective underwear of claim 10, further comprising a waterproof coating on the inside surface of the shorts.

15. The protective underwear of claim 10, wherein the fasteners comprise hook and loop fasteners.

16. The protective underwear of claim 10, wherein the liner is configured for removal from the shorts while the shorts are worn by the user wearing while the pants are pulled down but not removed from the user.
